(19) United States
(12) Patent Application Publication
(10) Pub. No.: US 2011/0064236 A1
(43) Pub. Date: Mar. 17, 2011

(54) ELECTRONIC STETHOSCOPE WITH NARROW PICKUP BEAM

(76) Inventor: Theodor Schubach, Miamisburg, OH (US)

(21) Appl. No.: 12/561,227

(22) Filed: Sep. 16, 2009

(51) Int. Cl. A61B 7/04

(52) U.S. Cl. 381/67

(57) ABSTRACT

An electronic stethoscope having a pickup head bearing a microphone, a sound system for transmitting sounds obtained from the microphone, and two earpieces each having a transducer for generating acoustic signals corresponding to the sounds picked up by the microphone. The electronic stethoscope may have electrical circuitry, an onboard battery connected to the electrical circuitry, an on-off switch controlling power from the battery, and a volume control feature for adjusting sound levels heard by the user. The electronic stethoscope may have an onboard sound recorder for recording the acoustic signals.

### FIELD OF THE INVENTION

The present invention pertains to electrical or electronic stethoscopes, and more particularly to an electrical or electronic stethoscope which uses a single microphone utilizing narrow pickup beam technology.

### BACKGROUND OF THE INVENTION

Electrical or electronic stethoscopes, which will be referred to hereinafter as electronic stethoscopes, have been proposed for diagnosing medical conditions. An electronic stethoscope uses a microphone to pick up acoustic inputs for subsequent diagnosis.

An important element of acoustic inputs is limitation of disruptive, unrelated noises from the environment. Various approaches have been developed to overcome disruptive environmental noises, such as suppression and filtering. An electronic stethoscope using two microphones has been proposed. The two microphones are movable so as to be able to approach and diverge from one another for focusing on a desired sound source area of the body.

### SUMMARY OF THE INVENTION

The present invention provides an electronic stethoscope which utilizes a single microphone of a type having narrowly focused pickup beam characteristics. The novel stethoscope can obtain and transmit sounds obtained from very limited portions of the body, thereby eliminating or minimizing unrelated sounds which might interfere with acoustic characteristics of a sought organ or body area. The narrow pickup beam enables the head of the novel stethoscope to be tilted relative to the skin, so that for example, a pickup beam can be obtained from an organ or body area from which direct access or acoustic travel path is interrupted by an intervening entity or organ. Illustratively, the head of the novel stethoscope may be positioned below or above a rib, and may be inclined so as to pick up sounds from an area directly behind the rib, thereby overcoming acoustic obstruction by the intervening member.

### BRIEF DESCRIPTION OF THE DRAWINGS

Similar reference characters denote corresponding features consistently throughout the attached drawings.

FIG. 1 is a perspective view of an electronic stethoscope according to at least one aspect of the invention.

FIG. 2 is an electrical schematic showing in simplified form electrical components of the stethoscope of FIG. 1 and electrical connections thereof.

FIG. 3 is a perspective view of an electronic stethoscope according to a further aspect of the invention.

### DETAILED DESCRIPTION

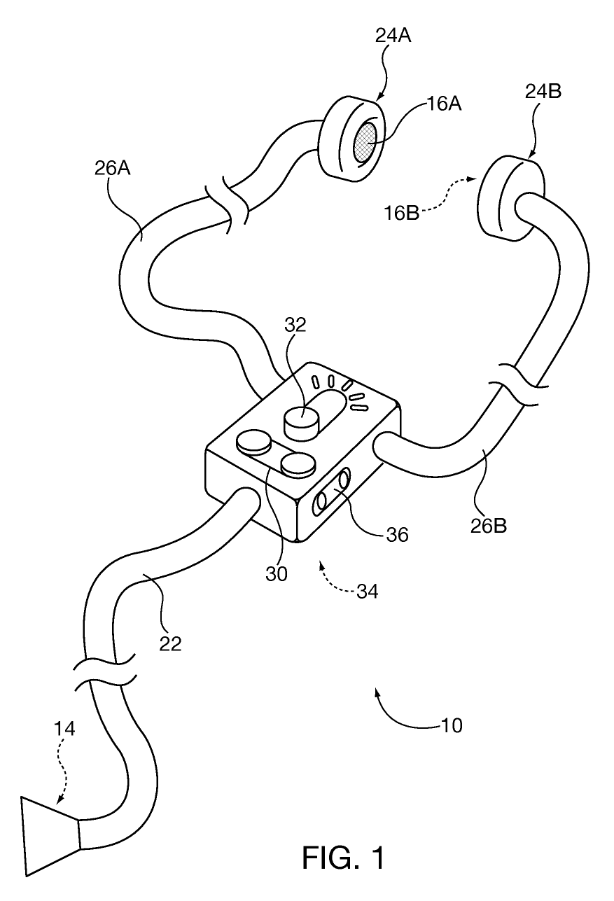

The present invention is seen in overview in FIG. 1 to comprise outwardly a stethoscope 10 having a pickup head or microphone 14 for acquiring sounds to be transmitted to the ears of the user (not shown). The microphone 14 may be of a type characterized by a narrow sound pickup beam, for obtaining acoustic inputs. The microphone 14 may be a horn microphone, may incorporate a parabolic reflector, may use directional filtering, or may be of any other known type which enables a single microphone to respond to a narrow sound pickup beam.

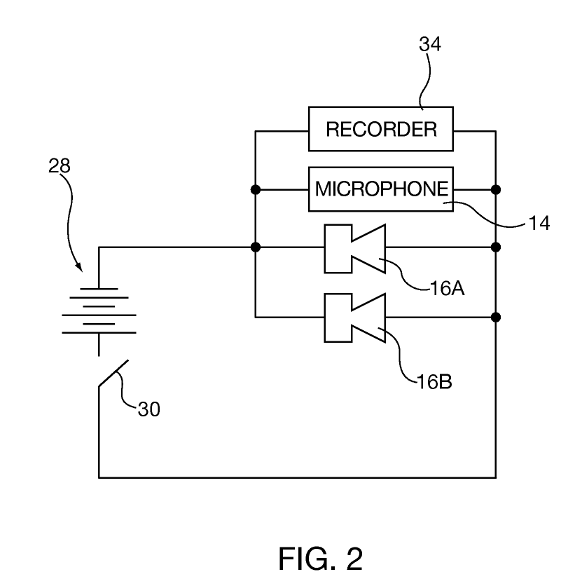

Referring also to FIG. 2, the novel stethoscope 10 is an electrical or electronic stethoscope since it uses an electrically powered sound system which produces signals responsive to the acoustic inputs. More specifically, the stethoscope 10 utilizes an input transducer (i.e., the microphone 14) to generate signals from acoustic inputs, and in turn to reproduce originally captured sounds from the input transducer using output transducers or speakers 16A, 16B. An electrical circuit generally indicated at 18 transmits signals generated by the input transducer or microphone 14 to the speakers 16A, 16B. Each speaker 16A or 16B is contained within a respective earpiece 20A or 20B.

In its physical configuration, the stethoscope 10 may generally resemble an ordinary pneumatic stethoscope (not shown, having an elongated flexible pickup tube 22, two earpieces 24A, 24B which contain the speakers 16A, 16B respectively, and two elongated flexible speaker tubes 26A, 26B which extend to the earpieces 24A, 24B.

The electrical circuit 18 comprises an onboard battery cell or cells, which will be referred to as a battery 28. The battery 28 is connected to the electrical circuit 18 so as to provide electrical power to the electrically powered components, such as the microphone 14 and the speakers 16A, 16B. The electrical circuit 18 includes an on-off switch 30 disposed to control electrical supply communication between the battery 28 and the rest of the electrical circuit 18.

A volume control feature disposed to selectively adjust sound volume of the acoustic signals may be incorporated into the electrical circuit 18. For example, a variable voltage switch 32 may control power to the speakers 16A, 16B.

The stethoscope 10 may comprise an onboard sound recorder 34 for recording the acoustic signals. Where provided, the onboard sound recorder 34 is powered by the battery 28 by suitable connection to the electrical circuit 18. The onboard sound recorder 34 may have operating functions of for example known tape recorders and other recording devices (none shown), such as an on-off control, volume control, playback control, and the like. The onboard sound recorder 34 may have manual controls 36. Suitable controls (not shown) may be provided as part of the onboard sound recorder 34 or may be separately provided.

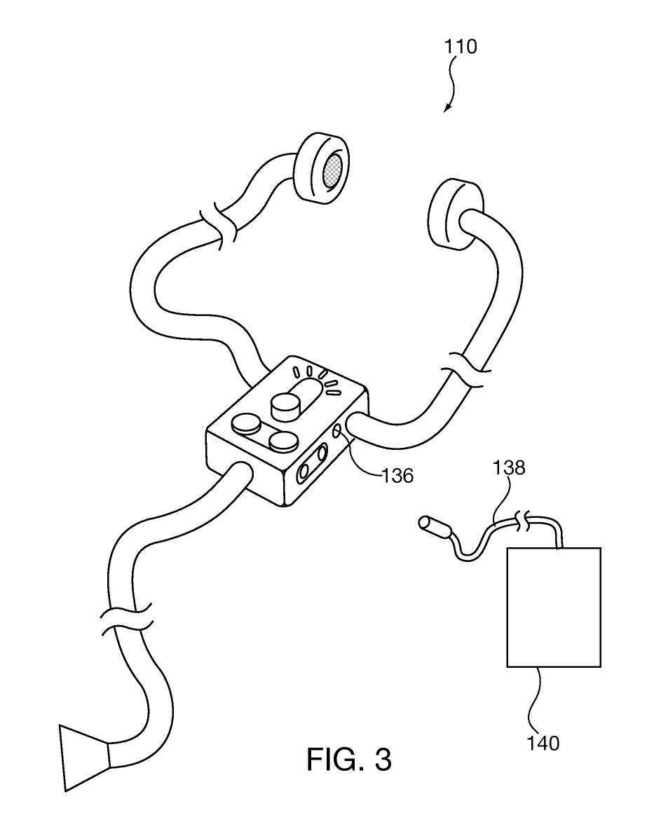

FIG. 3 shows an option wherein a stethoscope 110 according to a further aspect of the invention has a recorder 134 which is separate or detachable from the rest of the stethoscope 110. In all other respects, the stethoscope 110 may be the structural and functional equivalent of the stethoscope 10 of FIG. 1. The stethoscope 110 may have a signal port 136 to which a connection such as a communications cable 138 of a separate recording device 140 may be connected to record acquired sounds. This option enables a user to purchase the stethoscope 110 at less cost than that of the stethoscope 10, if the user saw no need for a recorder.

While the present has been described in connection with what is considered the most practical and preferred embodiments, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. An electronic stethoscope comprising:
a pickup head comprising a microphone of a type characterized by a narrow sound pickup beam, for obtaining acoustic inputs;
a sound system which produces signals responsive to the acoustic inputs, including an electrical circuit disposed to transmit the signals; and
two earpieces each having a transducer for generating acoustic signals from the signals obtained from the acoustic inputs.

2. The electronic stethoscope of claim 1, further comprising an onboard battery connected to the circuit and an on-off switch disposed to control electrical supply communication between the battery and the electrical circuit.

3. The electronic stethoscope of claim 1, further comprising a volume control feature disposed to selectively adjust sound volume of the acoustic signals.

4. The electronic stethoscope of claim 1, further comprising an onboard sound recorder for recording the acoustic signals.

5. An electronic stethoscope comprising:
a pickup head comprising a microphone, for obtaining acoustic inputs;
a sound system which produces signals responsive to the acoustic inputs, including an electrical circuit disposed to transmit the signals;
two earpieces each having a transducer for generating acoustic signals from the signals obtained from the acoustic inputs; and
an onboard sound recorder for recording the acoustic signals.

6. An electronic stethoscope comprising:
a pickup head comprising a microphone, for obtaining acoustic inputs;
a sound system which produces signals responsive to the acoustic inputs, including an electrical circuit disposed to transmit the signals;
two earpieces each having a transducer for generating acoustic signals from the signals obtained from the acoustic inputs; and
a port coupled to the sound system, disposed to transmit acoustic signals to a separate sound recorder.
